(19) United States
(12) Patent Application Publication
(10) Pub. No.: US 2019/0077842 A1
(43) Pub. Date: Mar. 14, 2019

(54) Method For Producing A Recombinant Protein With Reduced Impurities

(71) Applicant: Sandoz AG, Basel (CH)

(72) Inventor: Stefan Hutwimmer, Kundl (AT)

(73) Assignee: Sandoz AG, Basel (CH)

(21) Appl. No.: 15/767,246

(22) PCT Filed: Oct. 19, 2016
(86) PCT No.: PCT/EP2016/075040

§ 371 Date: Apr. 10, 2018

(30) Foreign Application Priority Data
Oct. 19, 2015 (EP) 15190359.8

(51) Int. Cl. C07K 14/535; A61P 7/00; C12N 15/70; C07K 1/14

(52) U.S. Cl. CPC C07K 14/535; C07K 1/14; C12N 15/70; A61P 7/00

(57) ABSTRACT

The present invention relates in general to a method of producing a recombinant protein, in particular hG-CSF, with reduced impurities resulting from truncation of said recombinant protein. The present invention also relates to a composition comprising a protein obtained with the inventive method.

The present invention relates to a method of producing a recombinant protein, in particular hG-CSF, with reduced impurities resulting from truncation of said recombinant protein. The present invention also relates to a composition comprising a protein obtained with the inventive method.

The biotechnological production of pharmaceutically relevant proteins brings about various challenges for the skilled person, inter alia due to the high regulatory standards for the purity of the pharmaceutically active protein. In order to achieve a level of purity complying with these standards, recombinantly produced proteins usually need to be purified after the initial isolation from the host cells. This process of removing impurities may involve several different steps. However, while each purification step improves the purity of the protein product, each purification step also brings about a loss in yield.

An example for recombinant production of a pharmaceutically active protein is granulocyte colony stimulating factor (G-CSF). G-CSF is a polypeptide based hormone of mammals. It is a cytokine and stimulates inter alia the production of granulocytes. G-CSF also stimulates the survival, proliferation, differentiation, and function of neutrophil precursors and mature neutrophils. The natural human glycoprotein exists in two forms, a (more active) 174- and (less active) 177-amino-acid-long polypeptide.

The 174 amino acid long version of human G-CSF (hG-CSF) has been used for several pharmaceutical applications. In oncology and hematology, hG-CSF is used with certain cancer patients to accelerate recovery from neutropenia (i.e. abnormally low number of neutrophils) after chemotherapy. G-CSF is also used to increase the number of hematopoietic stem cells in the blood of the donor before collection for use in hematopoietic stem cell transplantation. Several other clinical applications are contemplated as well.

U.S. Pat. No. 4,810,643 disclosed the recombinant expression of hG-CSF in prokaryotic or eukaryotic host cells. The resulting protein products displayed the physical and immunological properties and in vitro biological activities of isolates of hG-CSF derived from natural sources. G-CSF was first marketed by Amgen with the brand name Neupogen®. In 2014, the sales of Neupogen® amounted to about 1.2 billion US dollar worldwide. Several bio-generic versions are also available. The recombinant human G-CSF, synthesized in an E. coli expression system, is called filgrastim. The structure of filgrastim differs slightly from the structure of the natural glycoprotein, because it exhibits an additional methionine residue at the N-terminus and is not glycosylated. A pegylated version of filgrastim is also marketed. hG-CSF expressed in a mammalian expression systems (lenograstim), such as CHO cells, is indistinguishable from the 174-amino acid long natural (i.e. non-recombinant) human G-CSF.

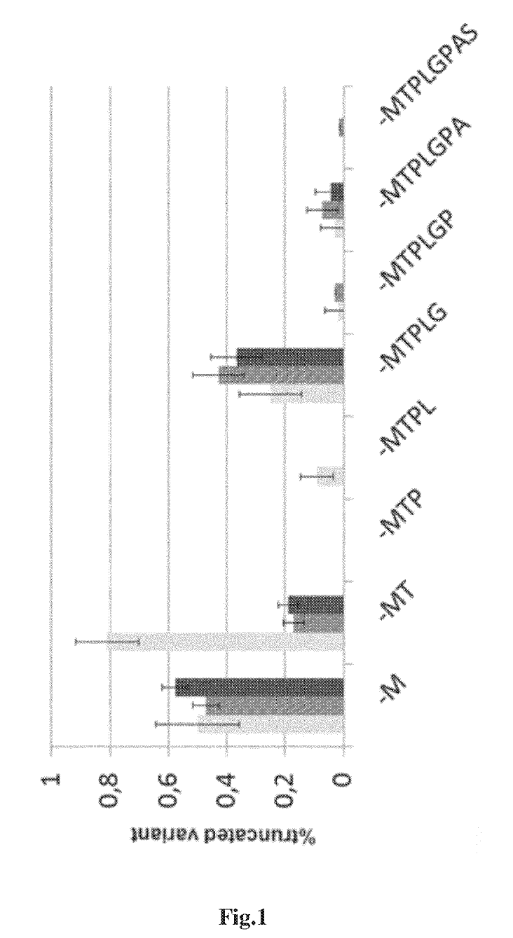

One problem arising in the biotechnological production of G-CSF is N-terminal truncation. G-CSF contains a non-structured, flexible N-terminal region of about 10 amino acids length which is prone to degradation. The amount of respective truncation byproducts must be reduced to meet the purity specifications for the pharmaceutical product. Said purification process in turn brings about a reduction in yield and concomitantly an increase in production costs. Characterization of three commercially available Filgrastim products reveals still residual presence of said truncation variants (see FIG. 1).

Given the loss in yield and the risk of failing regulatory requirements, there is thus a need in the art to establish new means for reducing said loss in yield and risk. It was thus the object of the present invention to provide a means for reducing the amount of truncation byproducts of biotechnologically produced proteins, for instance for reducing N-terminal truncation products of biotechnologically produced G-CSF.

This problem is solved by the subject-matter as set forth below and in the appended claims.

In the following a brief description of the appended figures will be given. The figures are intended to illustrate the present invention in more detail. However, they are not intended to limit the scope of the invention to these specific examples.

FIG. 1: illustrates the results of three different commercially available Filgrastim products with respect to presence of N-terminally truncated variants of G-CSF in the product. The analysis revealed that even in the final products there were still truncated versions of recombinant G-CSF present, which lacked up to 8 amino acids at the N-terminus.

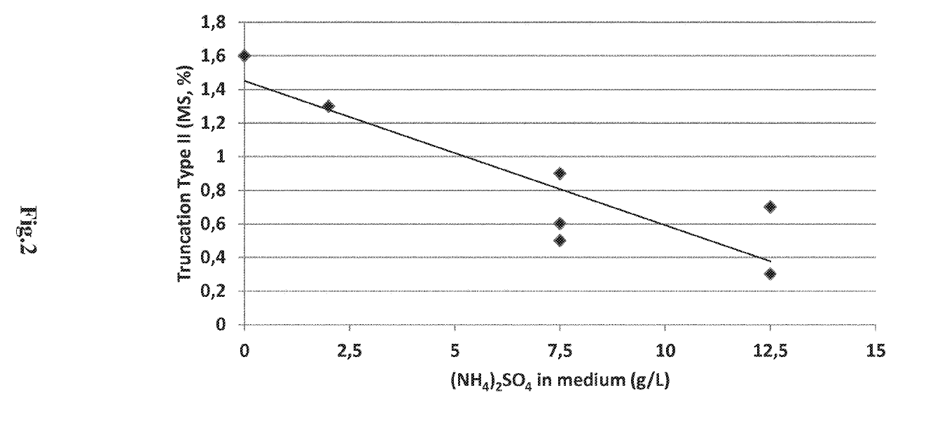

FIG. 2: is a graph comparing the abundance (%) of N-terminally truncated G-CSF species lacking the first 3 to 7 amino acids of recombinant human G-CSF (group II truncations) depending on the concentration of (NH₄)₂SO₄ used. The results are based on LC-MS-analysis.

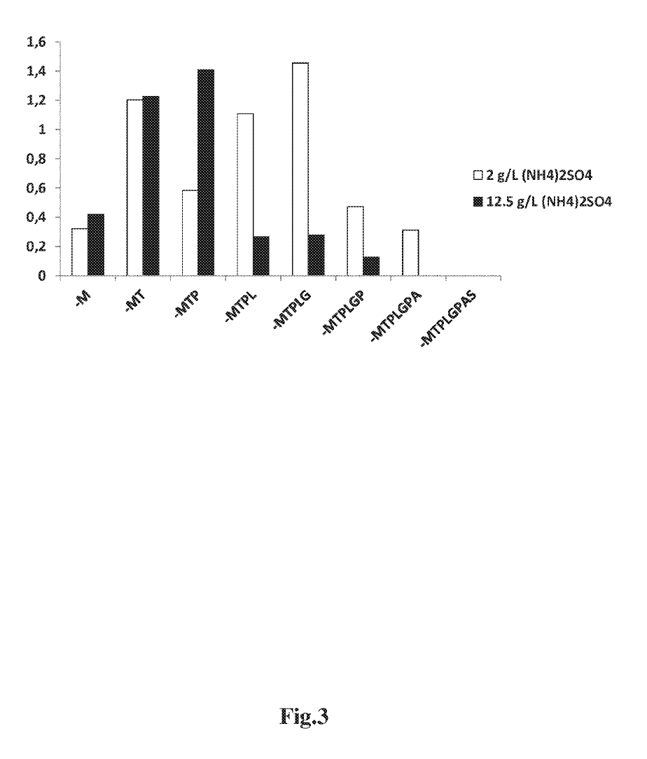

FIG. 3: is a graph comparing the abundance (%) of individual species of N-terminally truncated versions (lacking the first 1 to 8 amino acids) of recombinant human G-CSF depending on the concentration of (NH₄)₂SO₄ used. The results are based on LC-MS-analysis.

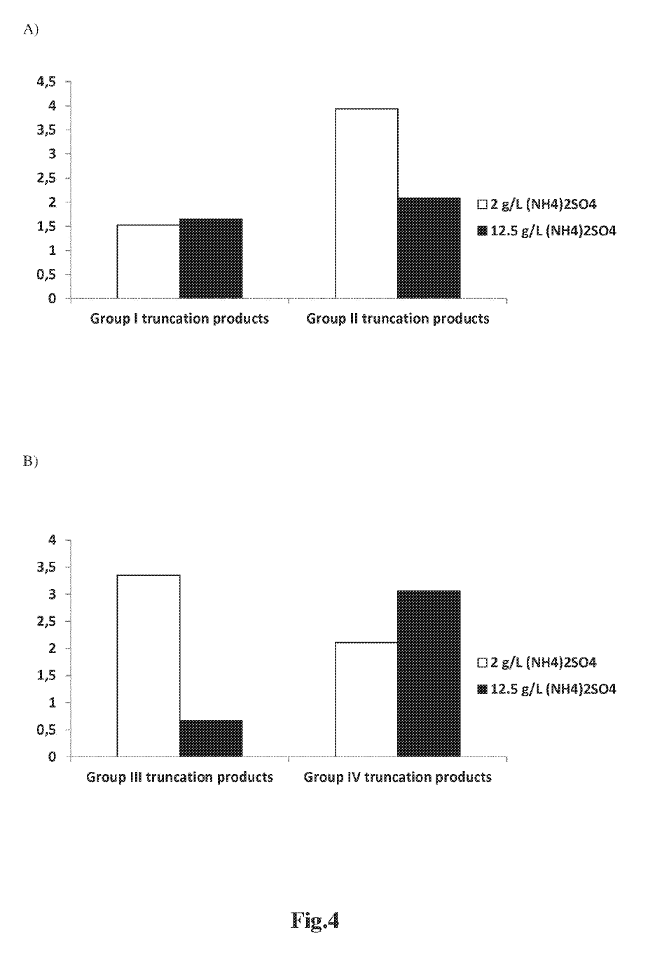

FIG. 4: is a graph comparing in A) the abundance (%) of N-terminally truncated versions lacking the first 1 or 2 amino acids of recombinant human G-CSF (group I truncations) vs. N-terminally truncated versions lacking the first 3 to 7 amino acids of recombinant human G-CSF (group II truncations) and in B) the abundance of N-terminally truncated versions lacking the first 1 to 3 amino acids of recombinant human G-CSF (group IV truncations) vs. N-terminally truncated versions lacking the first 4 to 7 amino acids of recombinant human G-CSF (group III truncations).

In a first aspect the present invention relates to a method of producing a protein with reduced impurities resulting from truncation of said protein, the method comprising the steps of:
a) culturing host cells expressing said protein in a culture medium in presence of a nitrogen source, wherein said nitrogen source is present in a concentration above standard,
b) isolating the protein, and
c) optionally purifying said isolated protein.

The inventor of the present invention has surprisingly found that truncated byproducts of a given biotechnologically produced protein, here exemplified for N-terminal truncation of G-CSF, can be reduced, if the levels of the nitrogen source (or of several nitrogen sources) in the culture media of the host cells is increased.

A protein produced with the method of the present invention is a protein, which is prone to truncation (in particular N-terminal truncation), if produced under standard culture conditions with respect to the nitrogen source used. A protein produced with the method of the present invention is preferably a recombinant protein, i.e. is encoded in the host cell by recombinant nucleic acid sequences. Furthermore, the produced protein is preferably heterologous to the producing host cell. The protein may for example be of mammalian origin, in particular of human origin, while the host cell is a prokaryotic cell, e.g. an E. coli cell. Usually (but not limited thereto) a protein produced by the method according to the present invention will be at least 30, at least 40, at least 50, at least 60, at least 70, at least 80, at least 90, at least 100, at least 110, at least 120, at least 130, at least 140, at least 150 or more than 160 amino acids long.

A particularly preferred protein to be produced with the method of the present invention is granulocyte colony stimulating factor (G-CSF). As used herein, the term “granulocyte colony stimulating factor”, or G-CSF, encompasses all forms of G-CSF known or conceivable for a person skilled in the art. The G-CSF may, for example, be of any mammalian origin, such as human G-CSF (hG-CSF), which is particularly preferred, mouse G-CSF (mG-CSF), or bovine G-CSF (bG-CSF). The term encompasses all allelic variants. The term encompasses recombinant G-CSF (i.e. with methionine at the N-terminus), for instance recombinant human G-CSF (SEQ ID NO: 1), as well as natural G-CSF (i.e. without methionine at the N-terminus), for instance natural human G-CSF (SEQ ID NO: 2). The sequence of SEQ ID NO: 3 represents a “generic” G-CSF sequence encompassing natural as well as recombinant human G-CSF. The term “G-CSF” does also encompass mutated versions of naturally occurring G-CSF. Preferably, such mutated versions of G-CSF do still comprise the flexible N-terminal region of about 10 amino acids length of G-CSF. Most preferably, such mutated versions do still exhibit the biological activity of G-CSF. Preferably, such mutated versions of naturally occurring G-CSF are at least 30, at least 40, at least 50, at least 60, at least 70, at least 80, at least 90, at least 100, at least 110, at least 120, at least 130, at least 140, at least 150 or more than 160 amino acids long. The nucleic acid encoding the granulocyte colony stimulating factor (G-CSF) may also encode a G-CSF precursor exhibiting a signal peptide at the N-terminus which is then posttranslationally proteolytically cleaved, yielding the actual protein product. The term G-CSF also encompasses fusion proteins comprising G-CSF on the one hand and one or more further fusion partners on the other hand. Particularly preferred are fusion proteins, in which G-CSF forms the N-terminal part of the fusion protein. Examples for potential fusion partners are, without being limited thereto, conventionally used tags, such as His-tags, or detectable markers such as GFP. The eventually produced G-CSF may be glycosylated, may be pegylated, may be both (i.e. is glycosylated and pegylated) or may be none of it (i.e. is neither glycosylated nor pegylated).

“Impurities resulting from truncation of said protein” are protein species deriving from the produced protein, e.g. G-CSF, but lacking one or more amino acids at the N- and/or C-terminus. Preferably, these protein species are N-terminally truncated vis-à-vis the full length protein, i.e. lack amino acids at the N-terminus. Most preferably, said impurities lack 1 to 7 N-terminal amino acids in comparison to the full length of the protein of interest.

If the method of the present invention is used to produce G-CSF, then the reduced impurities resulting from truncation of said protein are preferably G-CSF impurities resulting from group II truncation products of said G-CSF and/or resulting from group III truncation products of said G-CSF. As mentioned previously, G-CSF contains a non-structured, flexible N-terminal region of about 10 amino acids length which is prone to degradation. The present invention classifies the N-terminally truncated G-CSF products into various truncation subgroups: Group I truncation products of G-CSF, group II truncation products of G-CSF, group III truncation products of G-CSF, and group IV truncation products of G-CSF.

“Group I truncation products of G-CSF” are those N-terminally truncated G-CSF products, which still exhibit at least one amino acid residue N-terminal of the first leucine residue occurring on the N-terminal end of G-CSF. “The first leucine residue occurring on the N-terminal end”, as used herein, is necessarily a relative expression. The precise position of the leucine residue cannot be defined more precisely given the fact that the term “G-CSF” as used herein encompasses various entities, not all of which have the identical sequence of amino acid residues. The term “first leucine residue occurring on the N-terminal end” refers typically to the leucine residue occurring at position 3 of the G-CSF sequence (L3), see for example natural human (SEQ ID NO: 2), bovine, or mouse G-CSF. In recombinant G-CSF, where an additional methionine residue is present at the N-terminus, it corresponds to position 4 (see SEQ ID NO: 1). In G-CSF variants with additional amino acid residues N-terminal of the actual G-CSF sequence, the absolute position vis-à-vis the N-terminal end may be different. For example, in the non-cleaved G-CSF precursor including the signal peptide, it corresponds to position 33 (see for example SEQ ID NO: 4), because the term refers to the first leucine residue of G-CSF and not to the first leucine of the precursor sequence. In fusion proteins comprising G-CSF, in which G-CSF does not form the most N-terminal portion, the absolute position of the leucine residue will likewise be distinct and depends on the position of G-CSF within the fusion. However, a person skilled in the art will be readily capable of determining the position of the G-CSF sequence within such fusion protein and then the position of the leucine residue in question, e.g. by performing respective alignments of the sequence of the fusion protein and, e.g., the natural G-CSF sequence.

For natural human G-CSF (SEQ ID NO: 2) there is only one truncated polypeptide entity falling within the definition of “Group I truncation products of G-CSF”, namely truncated G-CSF lacking the first amino acid of natural human G-CSF: T (threonine). The Group I truncation product of G-CSF according to SEQ ID NO: 2 is thus truncated by one amino acid at the N-terminus. For recombinant human G-CSF (SEQ ID NO: 1) there are two truncation species falling under the definition, namely the polypeptide species lacking the N-terminal methionine residue of recombinant human G-CSF as well as the polypeptide species lacking the N-terminal methionine and threonine residues of recombinant human G-CSF. The Group I truncation products of G-CSF according to SEQ ID NO: 1 are thus truncated by one or two amino acids at the N-terminus. With respect to G-CSF of SEQ ID NO: 3, said “group I truncations” lack the N-terminal residues (M) or (M)T of SEQ ID NO: 3. “(M)” in brackets is intended to reflect that SEQ ID NO: 3 anyway allows absence of the N-terminal methionine. The Group I truncation products of G-CSF according to SEQ ID NO: 3 are thus truncated by one or two amino acids at the N-terminus.

In contrast to “group I truncation products of G-CSF”, “group II truncation products of G-CSF” lack up to 5 further amino acid residues (and not more) at the N-terminus of G-CSF. In other words, they lack all amino acids N-terminal of the first leucine residue occurring on the N-terminal end of G-CSF and lack 0 to 4 further amino acid residues (and not more) of the N-terminal amino acid residues of G-CSF.

With respect to natural human G-CSF (SEQ ID NO: 2), said “group II truncations” thus may lack the N-terminal residues TP (SEQ ID NO: 5), TPL (SEQ ID NO: 6), TPLG (SEQ ID NO: 7), TPLGP (SEQ ID NO: 8), or TPLGPA (SEQ ID NO: 9) of SEQ ID NO: 2. The group II truncation products of G-CSF according to SEQ ID NO: 2 are thus truncated by two, three, four, five or six amino acids at the N-terminus. With respect to recombinant human G-CSF (SEQ ID NO: 1) this means that said “group II truncations” are truncated versions of recombinant G-CSF lacking the N-terminal sequence motifs MTP (SEQ ID NO: 10), MTPL (SEQ ID NO: 11), MTPLG (SEQ ID NO: 12), MTPLGP (SEQ ID NO: 13), or MTPLGPA (SEQ ID NO: 14) of SEQ ID NO: 1. The Group II truncation products of G-CSF according to SEQ ID NO: 1 are thus truncated by three, four, five, six or seven amino acids at the N-terminus. With respect to G-CSF of SEQ ID NO: 3, said “group II truncations” lack the N-terminal residues (M)TP (SEQ ID NO: 15), (M)TPL (SEQ ID NO: 16), (M)TPLG (SEQ ID NO: 17), (M)TPLGP (SEQ ID NO: 18), or (M)TPLGPA (SEQ ID NO: 19) of SEQ ID NO: 3. “(M)” is intended to reflect that SEQ ID NO: 3 anyway allows absence of the N-terminal methionine. The Group II truncation products of G-CSF according to SEQ ID NO: 3 are thus truncated by three, four, five, six or seven amino acids at the N-terminus.

“Group III truncation products of G-CSF”, as used herein, represent a subgroup within “group II truncation products of G-CSF”. They always lack all amino acids N-terminal of the first leucine residue plus at least said leucine residue. In other words, they lack all amino acids N-terminal of the first leucine residue occurring on the N-terminal end of G-CSF and lack 1 to 4 further amino acid residues (and not more) of the N-terminal amino acid residues of G-CSF.

With respect to natural human G-CSF (SEQ ID NO: 2), said “group III truncations” thus lack the N-terminal residues TPL, TPLG, TPLGP, or TPLGPA of SEQ ID NO: 2. The group III truncation products of G-CSF according to SEQ ID NO: 2 are thus truncated by three, four, five or six amino acids at the N-terminus. With respect to recombinant human G-CSF (SEQ ID NO: 1) this means that said “group III truncations” are truncated versions of recombinant G-CSF lacking the N-terminal sequence motifs MTPL, MTPLG, MTPLGP, or MTPLGPA of SEQ ID NO: 1. The Group III truncation products of G-CSF according to SEQ ID NO: 2 are thus truncated by four, five, six or seven amino acids at the N-terminus. With respect to G-CSF of SEQ ID NO: 3, said “group III truncations” lack the N-terminal residues (M)TPL, (M)TPLG, (M)TPLGP, or (M)TPLGPA of SEQ ID NO: 3. “(M)” is intended to reflect that SEQ ID NO: 3 anyway allows absence of the N-terminal methionine. The Group III truncation products of G-CSF according to SEQ ID NO: 3 are thus truncated by four, five, six or seven amino acids at the N-terminus.

“Group IV truncation products of G-CSF”, as used herein, comprise “Group I truncation products of G-CSF” plus the truncation product lacking all amino acids N-terminal of the first leucine residue. Group IV truncation products of G-CSF do still exhibit said leucine residue.

For natural human G-CSF (SEQ ID NO: 2) there are two truncated polypeptide entities falling within the definition of “group IV truncation products of G-CSF”, namely truncated G-CSF lacking the first amino acid of natural human G-CSF: T (threonine); and truncated G-CSF lacking the first amino acid and the second amino acid of natural human G-CSF: TP. The group IV truncation products of G-CSF according to SEQ ID NO: 2 are thus truncated by one amino acid or two amino acids at the N-terminus. For recombinant human G-CSF (SEQ ID NO: 1) there are three truncation species falling under the definition, namely truncated versions of recombinant G-CSF lacking the N-terminal sequence motifs M, MT or MTP. The group IV truncation products of G-CSF according to SEQ ID NO: 1 are thus truncated by one, two or three amino acids at the N-terminus. With respect to G-CSF of SEQ ID NO: 3, said “group IV truncations” lack the N-terminal residues (M), (M)T or (M)TP of SEQ ID NO: 3. “(M)” in brackets is intended to reflect that SEQ ID NO: 3 anyway allows absence of the N-terminal methionine. The group IV truncation products of G-CSF according to SEQ ID NO: 3 are thus truncated by one, two or three amino acids at the N-terminus.

The table below illustrates the classification of “group I truncation products of G-CSF”, “group II truncation products of G-CSF”, “group III truncation products of G-CSF” and “group IV truncation products of G-CSF” for human recombinant G-CSF with respect to the truncated (i.e. missing) residues (left) as well as for the resulting N-terminal sequence of the truncated product.

| | | |
|---|---|---|
| trunc. | Group | N-Terminus of |

| residues | I | II | III | IV | fragment |
|---|---|---|---|---|---|
| -M | + | − | − | + | TPLGPAS . . . |
| -MT | + | − | − | + | PLGPAS . . . |
| -MTP | − | + | − | + | LGPAS . . . |
| -MTPL | − | + | + | − | GPAS . . . |
| -MTPLG | − | + | + | − | PAS . . . |
| -MTPLGP | − | + | + | − | AS . . . |
| -MTPLGPA | − | + | + | − | S . . . |

The concepts of “group I truncation products of G-CSF”, “group II truncation products of G-CSF”, “group III truncation products of G-CSF” and “group IV truncation products of G-CSF” have been illustrated above for the sequence of human G-CSF. However, a person skilled in the art will readily be capable of applying said concepts to G-CSF of other origin (e.g. other mammalian origin such as bovine or murine G-CSF, G-CSF with point mutations in said stretch etc.,) as well, where the individual amino acid sequence at the N-terminus of said G-CSF may be individually distinct from the human sequence.

It is understood that truncated G-CSF products lacking more amino acid residues at the N-terminus than specified for “group I truncation products of G-CSF”, “group II truncation products of G-CSF”, “group III truncation products of G-CSF” or “group IV truncation products of G-CSF”, respectively, do not fall within the definition of the respective groups.

The host cells used according to the method of the present invention may be any type of host cell suited for (e.g. recombinant) protein production. The host cell may be a mammalian cell, such as a CHO cell, but may also be a bacterial cell such as an E. coli cell. E. coli cells are particularly preferred, if the produced protein is recombinant G-CSF, such as recombinant human G-CSF. In order to enable production of the protein of interest, e.g. G-CSF, a host cell may comprise a respective nucleic acid encoding the protein of interest. Said nucleic acid may further comprise elements operably linked to the sequence encoding said protein, which allow the transcription of the nucleic acid sequence and translation of the resulting mRNA into the encoded protein in the given host cell. In particular, the nucleic acid may comprise a heterologous promoter. Said heterologous promoter may be operably linked to the nucleic acid sequence encoding the protein, e.g. granulocyte colony stimulating factor (G-CSF), thereby providing for transcription of said nucleic acid sequence. A “heterologous promotor” for the nucleic acid encoding the protein of interest is a promoter, that is not found in direct association with the respective nucleic acid sequence encoding said protein in nature, i.e. is in nature not operably linked with the respective nucleic acid sequence encoding said protein.

A culture medium, as used herein, is any type of medium sustaining the growth of cells. The choice of the culture medium will depend on the choice of host cells for the production to the protein of interest. A person skilled in the art of cell culture and fermentation is readily aware of a number of suitable media for the respective host cells.

As mentioned above, the method according to the present invention requires that the host cells expressing the protein of interest are cultured in presence of a nitrogen source. The nitrogen source may be any nitrogen compound which can be utilized by the chosen host cell. The nitrogen source may be an organic nitrogen source but is preferably an inorganic nitrogen source. An example for a suitable nitrogen source is ammonium, in particular if provided in the form of ammonium salts, such as (NH₄)₂SO₄, (NH₄)H₂PO₄, (NH₄)₂HPO₄, (NH₄)₂Fe(SO₄)₂ 6.H₂O, CH₄N₂O, NH₄NO₃ or NH₄Cl. Another example is NH₃/NH₄OH. If the host cells are E. coli cells, then (NH₄)₂SO₄ is a particularly preferred nitrogen source.

As mentioned above, the method of the present invention utilizes the nitrogen source in a “concentration above standard”. This implies in particular that the person skilled in the art, confronted for example with the problem that the currently employed fermentation or cell culture method produces too many truncation products, needs to increase the concentration of the nitrogen source in said fermentation or cell culture method. The “standard concentration” for a given nitrogen source will of course be dependent on the actual nitrogen source chosen. However, such standard concentrations will be readily known to the person skilled in the art. For ammonium such standard concentration is considered to be for example a concentration of 1 to 1.2 g/L, in particular for E. coli cells. Standard concentrations for corresponding ammonium salts (e.g. (NH₄)₂SO₄ or (NH₄)₂HPO₄) in E. coli fermentation are for example disclosed in Riesenberg et al. (Appl Microbiol Biotechnol. 1990 October; 34(1):77-82) and Korz et al. (Journal of Biotechnology 39 (1995) 59-65). If the nitrogen source is ammonium (or comprises ammonium, respectively), then the concentration of said ammonium is in the inventive method preferably at least 70 mM, at least 80 mM, at least 90 mM, at least 100 mM, at least 110 mM, at least 120 mM, at least 130 mM, at least 140 mM, at least 150 mM, at least 160 mM, at least 170 mM, at least 180 mM, at least 190 mM, at least 200 mM, at least 210 mM, at least 220 mM, at least 230 mM, at least 240 mM or even higher. These ranges apply in particular if the nitrogen source is NH₄Cl, (NH₄)H₂PO₄, or NH₄OH.

If the nitrogen source is (NH₄)₂SO₄, then the concentration of (NH₄)₂SO₄ is preferably at least 35 mM, at least 40 mM, at least 45 mM, at least 50 mM, at least 55 mM, at least 60 mM, at least 65 mM, at least 70 mM, at least 75 mM, at least 80 mM, at least 85 mM, at least 90 mM, at least 95 mM, at least 100 mM, at least 105 mM, at least 110 mM, at least 115 mM, at least 120 mM or even higher.

A person skilled in the art will know that the increased concentration of the nitrogen source may be achieved by various means. For example, one may increase the levels of the nitrogen source in the medium prior to cultivating the host cells in said medium. One may for example increase the concentration of (NH₄)₂SO₄ in the medium. Alternatively (or even additionally), one may increase the NH₄OH concentration prior to sterilization when preparing the medium. If the final medium involves a complex nitrogen source, e.g. in the form of yeast autolysate, yeast extract, peptone etc. , then a nitrogen source such as (NH₄)₂SO₄ can also be added to said complex nitrogen source prior to preparation of the final medium. An alternative to increasing the nitrogen source concentration in the medium prior to cell cultivation (or in addition thereto) may be that the nitrogen source, e.g. (NH₄)₂SO₄ or NH₄OH is added in high concentrations to the ongoing fermentation or cell culture process (bolus addition). Such an addition can also be done repeatedly or even continuously to keep a high concentration of nitrogen source. Combinations of different routes to increase the concentration of the nitrogen source are also contemplated (e.g. higher start concentration of the nitrogen source in the medium plus later bolus addition in the fermenter).

It is particularly preferred if human G-CSF is produced with the method of the present invention, in particular recombinant human G-CSF according to SEQ ID NO: 1. The host cell is preferably a prokaryotic host cell, such as an E. coli host cell.

A method of the present invention may for instance involve culturing host cells, such as E. coli cells, expressing a protein, e.g. recombinant human G-CSF according to SEQ ID NO: 1, in a culture medium in presence of (NH₄)₂SO₄, wherein said (NH₄)₂SO₄ is present in a concentration above 2 g/L, such as at least 2.5 g/L, at least 3 g/L, at least 3.5 g/L, at least 4 g/L, at least 4.5 g/L, at least 5 g/L, at least 5.5 g/L, at least 6 g/L, at least 6.5 g/L, at least 7 g/L, at least 7.5 g/L, at least 8 g/L, at least 8.5 g/L, at least 9 g/L, at least 9.5 g/L, at least 10 g/L, at least 10.5 g/L, at least 11 g/L, at least 11.5 g/L, at least 12 g/L, at least 12.5 g/L, or even more.

The inventive method is a method of producing a protein, e.g. G-CSF, in particular recombinant G-CSF. The method comprises the step of culturing host cells expressing said recombinant protein. In this context, the actual type of production of said recombinant protein is not limited by the invention. The production method may for example foresee intracellular protein production, protein production in form of inclusion bodies, secretion of the produced protein into the periplasm, secretion of the protein of interest into the surrounding media etc.

The method according to the present invention involves isolating the produced protein from the respective host cells. A person skilled in the art is readily aware of various methods to do so. Unless the produced protein is exported to the surrounding medium, such procedure will most often involve lysis of the respective host cells. Usually, the thus isolated protein will require further purification, in particular if regulatory standards must be met. Various protein purifications techniques are known to the person skilled in the art. If the produced protein is G-CSF, such purification may for example involve cation exchange chromatography.

The present invention also relates to a composition comprising a protein of interest, such as G-CSF, said composition being obtainable or obtained by a method according to the present invention.

In a further aspect the present invention relates to a composition comprising G-CSF, wherein said G-CSF comprises less than 0.5%, in particular less than 0.4% (w/w) G-CSF impurities resulting from group II truncation products of said G-CSF. It is understood that wherever herein percentages of impurities resulting from truncation products of G-CSF are mentioned, that these percentages are given vis-à-vis the total content of G-CSF (non-truncated G-CSF+truncated impurities).

More preferably, the composition of the invention comprises less 0.38%, less than 0.36%, less than 0.35%, less than 0.34%, less than 0.32%, less than 0.3%, less than 0.28%, less than 0.26%, less than 0.25%, less than 0.24%, less than 0.22%, less than 0.20%, less than 0.18%, less than 0.16%, less than 0.15%, less than 0.12%, less than 0.10%, less than 0.08%, less than 0.06%, less than 0.05% or even 0.0% (i.e. below the detection limit of mass spectrometry) of said impurities resulting for group II truncation products of G-CSF.

In some embodiments, the composition of the present invention may comprise less than 0.5%, in particular less than 0.4% (w/w) G-CSF impurities resulting from group III truncation products of said G-CSF.

More preferably, the composition of the invention comprises less 0.38%, less than 0.36%, less than 0.35%, less than 0.34%, less than 0.32%, less than 0.3%, less than 0.28%, less than 0.26%, less than 0.25%, less than 0.24%, less than 0.22%, less than 0.20%, less than 0.18%, less than 0.16%, less than 0.15%, less than 0.12%, less than 0.10%, less than 0.08%, less than 0.06%, less than 0.05% or even 0.0% (i.e. below the detection limit of mass spectrometry) of said impurities resulting for group III truncation products of G-CSF.

The composition according to the present invention may comprises less than 0.3%, less than 0.28%, less than 0.26%, less than 0.25%, less than 0.24%, less than 0.22%, less than 0.20%, less than 0.18%, less than 0.16%, less than 0.15%, less than 0.12%, less than 0.10%, less than 0.08%, less than 0.06%, less than 0.05% or even 0.0% (i.e. below the detection limit of mass spectrometry) of the G-CSF truncation product lacking all amino acids N-terminal of the first leucine residue occurring on the N-terminal end of G-CSF and the next two N-terminal amino acids (including the leucine residue).

With respect to human G-CSF this implicates that the composition according to the present invention may comprise less than 0.3%, less than 0.28%, less than 0.26%, less than 0.25%, less than 0.24%, less than 0.22%, less than 0.20%, less than 0.18%, less than 0.16%, less than 0.15%, less than 0.12%, less than 0.10%, less than 0.08%, less than 0.06%, less than 0.05% or even 0.0% (i.e. below the detection limit of mass spectrometry) of a G-CSF truncation product lacking:
i) the N-terminal sequence motif TPLG for natural human G-CSF (SEQ ID NO: 2), i.e. lacking the first four N-terminal amino acids,
ii) the N-terminal sequence motif MTPLG for recombinant human G-CSF (SEQ ID NO: 1), i.e. lacking the first five N-terminal amino acids, or
iii) lacking the N-terminal sequence motif (M)TPLG for generic human G-CSF (SEQ ID NO: 3), i.e. lacking the first five N-terminal amino acids.

Likewise, the composition according to the present invention may comprise less than 0.10%, less than 0.09%, less than 0.08%, less than 0.07%, less than 0.06%, less than 0.05%, less than 0.04%, less than 0.03%, less than 0.02%, less than 0.01% or even 0.0% (i.e. below the detection limit of mass spectrometry) of a G-CSF truncation product lacking all amino acids N-terminal of the first leucine residue occurring on the N-terminal end of G-CSF and lacking the leucine residue.

With respect to human G-CSF this means that the composition according to the present invention may comprise less than 0.10%, less than 0.09%, less than 0.08%, less than 0.07%, less than 0.06%, less than 0.05%, less than 0.04%, less than 0.03%, less than 0.02%, less than 0.01% or even 0.0% (i.e. below the detection limit of mass spectrometry) of a G-CSF truncation product lacking:
i) the N-terminal sequence motif TPL for natural human G-CSF (SEQ ID NO: 2), i.e. lacking the first three N-terminal amino acids,
ii) the N-terminal sequence motif MTPL for recombinant human G-CSF (SEQ ID NO: 1), i.e. lacking the first four N-terminal amino acids, or
iii) the N-terminal sequence motif (M)TPL for generic human G-CSF SEQ ID NO: 3, i.e. lacking the first four N-terminal amino acids.

Likewise, the composition according to the present invention may comprises less than 0.10%, less than 0.09%, less than 0.08%, less than 0.07%, less than 0.06%, less than 0.05%, less than 0.04%, less than 0.03%, less than 0.02%, less than 0.01% or even 0.0% (i.e. below the detection limit of mass spectrometry) of a G-CSF truncation product lacking all amino acids N-terminal of the first leucine residue occurring on the N-terminal end of G-CSF and three further amino acids (including the leucine residue).

With respect to human G-CSF this means that the composition according to the present invention may comprise less than 0.10%, less than 0.09%, less than 0.08%, less than 0.07%, less than 0.06%, less than 0.05%, less than 0.04%, less than 0.03%, less than 0.02%, less than 0.01% or even 0.0% (i.e. below the detection limit of mass spectrometry) of a G-CSF truncation product lacking:
i) the N-terminal sequence motif TPLGP for natural human G-CSF (SEQ ID NO: 2), i.e. lacking the first five N-terminal amino acids,
ii) the N-terminal sequence motif MTPLGP for recombinant human G-CSF (SEQ ID NO: 1), i.e. lacking the first six N-terminal amino acids, or
iii) the N-terminal sequence motif (M)TPLGP for generic human G-CSF SEQ ID NO: 3, i.e. lacking the first six N-terminal amino acids.

As the present invention affects in particular the abundance of group II truncation products in the composition, the composition according to the present invention may for example still comprise group I truncation products of G-CSF, as defined herein. The composition of the present invention may thus comprise for example up to 0.5%, up to 0.6%, up to 0.7%, up to 0.8%, up to 0.9%, up to 1.0%, up to 1.1%, up to 1.2%, up to 1.3%, up to 1.4%, or even up to 1.5% or more G-CSF truncation products exhibiting at least one amino acid N-terminal of the first leucine residue occurring on the N-terminal end of G-CSF.

With respect to human G-CSF this means that the composition according to the present invention may comprise for example up to 0.5%, up to 0.6%, up to 0.7%, up to 0.8%, up to 0.9%, up to 1.0%, up to 1.1%, up to 1.2%, up to 1.3%, up to 1.4%, or even up to 1.5% or more G-CSF truncation products lacking:
i) the N-terminal amino acid T for natural human G-CSF (SEQ ID NO: 2), i.e. lacking the first N-terminal amino acid,
ii) the N-terminal sequence motifs M or MT for recombinant human G-CSF (SEQ ID NO: 1), i.e. lacking the first or the first and the second N-terminal amino acid, or
iii) the N-terminal sequence motif (M) or (M)T for generic human G-CSF (SEQ ID NO: 3), i.e. lacking the first or the first and the second N-terminal amino acid.

As the present invention affects within the group II truncation group in particular the abundance of group III truncation products in the composition, the composition according to the present invention may for example still comprise group IV truncation products of G-CSF, as defined herein. The composition of the present invention may thus comprise for example up to 0.5%, up to 0.6%, up to 0.7%, up to 0.8%, up to 0.9%, up to 1.0%, up to 1.1%, up to 1.2%, up to 1.3%, up to 1.4%, or even up to 1.5% or more of group IV truncation products.

With respect to human G-CSF this means that the composition according to the present invention may comprise for example up to 0.5%, up to 0.6%, up to 0.7%, up to 0.8%, up to 0.9%, up to 1.0%, up to 1.1%, up to 1.2%, up to 1.3%, up to 1.4%, or even up to 1.5% or more G-CSF truncation products lacking:
i) the N-terminal sequence motifs T or TP for natural human G-CSF (SEQ ID NO: 2), i.e. lacking the first or the first and the second N-terminal amino acid,
ii) the N-terminal sequence motifs M, MT, or MTP for recombinant human G-CSF (SEQ ID NO: 1), i.e. lacking the first one to three N-terminal amino acids, or
iii) the N-terminal sequence motif (M), (M)T or (M)TP for generic human G-CSF (SEQ ID NO: 3), i.e. lacking the first one to three N-terminal amino acids.

The composition of the present invention may also be characterized by the ratio of the abundance of group II truncation products within the G-CSF fraction of the composition to the abundance of group I truncation products within the G-CSF fraction. Said ratio of group II truncation products to group I truncation products may be less than 0.3, preferably less than 0.25, preferably less than 0.2, preferably less than 0.15, more preferably less than 0.1, more preferably less than 0.05, more preferably less than 0.025, more preferably less than 0.01 or may most preferably be even 0.

The composition of the present invention may also be characterized by the ratio of the abundance of group III truncation products within the G-CSF fraction of the composition to the abundance of group IV truncation products within the G-CSF fraction. Said ratio of group III truncation products to group IV truncation products may be less than 0.3, preferably less than 0.25, preferably less than 0.2, preferably less than 0.15, more preferably less than 0.1, more preferably less than 0.05, more preferably less than 0.025, more preferably less than 0.01 or may most preferably be even 0.

The composition of the present invention may also be characterized by the ratio of the abundance of the truncation product lacking all amino acids N-terminal of the first leucine residue occurring on the N-terminal end of G-CSF and two further amino acids (including the leucine residue) within the G-CSF fraction of the composition to the abundance of group I truncation products within the G-CSF fraction. Said ratio of said truncation product to group I truncation products may be less than 0.3, preferably less than 0.25, preferably less than 0.2, preferably less than 0.15, more preferably less than 0.1, more preferably less than 0.05, more preferably less than 0.025, more preferably less than 0.01 or may most preferably be even 0.

With respect to human G-CSF this means that the ratio of:
a) the abundance of a G-CSF truncation product lacking:
   i) the N-terminal sequence motif TPLG for natural human G-CSF (SEQ ID NO: 2), i.e. lacking the first four N-terminal amino acids,
   ii) the N-terminal sequence motif MTPLG for recombinant human G-CSF (SEQ ID NO: 1), i.e. lacking the first five N-terminal amino acids, or
   iii) the N-terminal sequence motif (M)TPLG for generic human G-CSF (SEQ ID NO: 3), i.e. lacking the first five N-terminal amino acids, to
b) the abundance of the respective group I truncation products of G-CSF, namely G-CSF truncation product lacking:
   iv) the N-terminal amino acid T for natural human G-CSF (SEQ ID NO: 2), i.e. lacking the first N-terminal amino acid,
   v) the N-terminal sequence motifs M and MT for recombinant human G-CSF (SEQ ID NO: 1), i.e. lacking the first or the first and the second N-terminal amino acid, or
   vi) the N-terminal sequence motifs (M) and (M)T for generic human G-CSF (SEQ ID NO: 3), i.e. lacking the first or the first and the second N-terminal amino acid,
   may be less than 0.25, more preferably less than 0.2, more preferably less than 0.15, more preferably less than 0.1, more preferably less than 0.05, more preferably less than 0.025, more preferably less than 0.01 or may most preferably be even 0.

To be clear: this means for instance for recombinant human G-CSF (SEQ ID NO: 1), that the ratio of

a) the abundance of a G-CSF truncation product lacking the N-terminal sequence motif MTPLG, i.e. lacking the first five N-terminal amino acids of recombinant human G-CSF (SEQ ID NO: 1), to

b) the abundance of the respective group I truncation products of recombinant human G-CSF, namely G-CSF truncation product lacking the N-terminal sequence motifs M and MT, i.e. lacking the first or the first and the second N-terminal amino acid of recombinant human G-CSF (SEQ ID NO: 1)

may be less than 0.25, more preferably less than 0.2, more preferably less than 0.15, more preferably less than 0.1, more preferably less than 0.05, more preferably less than 0.025, more preferably less than 0.01 or may most preferably be even 0.

A further ratio preferably (but not necessarily) characterising the composition of the present invention is within the G-CSF fraction of the composition the ratio between the abundance of the truncation product lacking all amino acids N-terminal of the first leucine residue, i.e. the truncation product with the leucine residue on the N-terminus, to the abundance of the truncation product lacking all amino acids N-terminal of the first leucine residue occurring on the N-terminal end of G-CSF and two further amino acids (including the leucine residue). Said ratio is preferably more than 0.5, preferably more than 0.6, preferably more than 0.7, preferably more than 0.8, preferably more than 0.9, preferably more than 1, preferably more than 1.2, preferably more than 1.4, preferably more than 1.6, preferably more than 1.8, preferably more than 2, preferably more than 2.5, preferably more than 3 or most preferably more than 4 or even higher.

With respect to human G-CSF this means that the ratio of:
a) the abundance of the truncation product lacking all amino acids N-terminal of the first leucine residue, namely G-CSF truncation product lacking:
   i) the N-terminal amino acid TP for natural human G-CSF (SEQ ID NO: 2), i.e. lacking the first two N-terminal amino acid,
   ii) the N-terminal sequence motif MTP for recombinant human G-CSF (SEQ ID NO: 1), i.e. lacking the first three N-terminal amino acids, or
   iii) the N-terminal sequence motifs (M)TP for generic human G-CSF (SEQ ID NO: 3), i.e. lacking the first three N-terminal amino acids, to
b) the abundance of a G-CSF truncation product lacking:
   iv) the N-terminal sequence motif TPLG for natural human G-CSF (SEQ ID NO: 2), i.e. lacking the first four N-terminal amino acids,
   v) the N-terminal sequence motif MTPLG for recombinant human G-CSF (SEQ ID NO: 1), i.e. lacking the first five N-terminal amino acids, or
   vi) the N-terminal sequence motif (M)TPLG for generic human G-CSF (SEQ ID NO: 3), i.e. lacking the first five N-terminal amino acids,
   may be preferably more than 0.5, preferably more than 0.6, preferably more than 0.7, preferably more than 0.8, preferably more than 0.9, preferably more than 1, preferably more than 1.2, preferably more than 1.4, preferably more than 1.6, preferably more than 1.8, preferably more than 2, preferably more than 2.5, preferably more than 3 or most preferably more than 4 or even higher.

This means for instance for recombinant human G-CSF (SEQ ID NO: 1), that the ratio of
a) the abundance of the truncation product lacking the N-terminal sequence motif MTP, i.e. lacking the first three N-terminal amino acids of recombinant human G-CSF (SEQ ID NO: 1), to
b) the abundance of a G-CSF truncation product lacking the N-terminal sequence motif MTPLG, i.e. lacking the first five N-terminal amino acids of recombinant human G-CSF (SEQ ID NO: 1),

may be preferably more than 0.5, preferably more than 0.6, preferably more than 0.7, preferably more than 0.8, preferably more than 0.9, preferably more than 1, preferably more than 1.2, preferably more than 1.4, preferably more than 1.6, preferably more than 1.8, preferably more than 2, preferably more than 2.5, preferably more than 3 or most preferably more than 4 or even higher.

The composition according to the present invention comprising G-CSF may be for example a cell lysate, in particular a cell lysate of a host cell according to the present invention. However, the composition according to the present invention is most preferably a pharmaceutical composition comprising G-CSF and a pharmaceutically acceptable carrier, diluent and/or excipient.

In a further aspect, the present invention relates to a composition according to the present invention, in particular a pharmaceutical composition according to the present invention, for use in a method for the treatment of the human or animal body by therapy. In particular, the present invention relates a composition according to the present invention comprising G-CSF, in particular a pharmaceutical composition according to the present invention comprising G-CSF, for use in the treatment or prevention of neutropenia.

In a further aspect, the present invention relates to method of treatment of a subject suffering from neutropenia, the method comprising the step of administering a pharmaceutical composition according to the present invention comprising G-CSF to said subject in an effective amount.

In a further aspect, the present invention relates to a method of stimulating the survival, proliferation, differentiation, and function of neutrophil precursors and mature neutrophils in a subject in need thereof, the method comprising the step of administering a pharmaceutical composition according to the present invention comprising G-CSF to said subject in an effective amount.

In a further aspect, the present invention relates to a method of increasing the number of hematopoietic stem cells in the blood of a subject, the method comprising the step of administering a pharmaceutical composition according to the present invention comprising G-CSF to said subject in an effective amount.

The term “comprising”, as used herein, shall not be construed as being limited to the meaning “consisting of” (i.e. excluding the presence of additional other matter). Rather, “comprising” implies that optionally additional matter, features or steps may be present. The term “comprising” encompasses as particularly envisioned embodiments falling within its scope “consisting of” (i.e. excluding the presence of additional other matter) and “comprising but not consisting of” (i.e. requiring the presence of additional other matter, features or steps), with the former being more preferred.

The use of the word “a” or “an”, when used herein, may mean “one,” but it is also consistent with the meaning of “one or more,” “at least one,” and “one or more than one.”

### EXAMPLES

In the following, specific examples illustrating various embodiments and aspects of the invention are presented. However, the present invention shall not to be limited in scope by the specific embodiments described herein. Indeed, various modifications of the invention in addition to those described herein will become readily apparent to those skilled in the art from the foregoing description, accompanying figures and the examples below. All such modifications fall within the scope of the appended claims.

### Example 1

### Analysis of the Abundance of N-Terminal G-CSF Truncation Products in Three Commercial Products of Recombinant hG-CSF

Nine micrograms of three filgrastim products were separated on a Zorbax 300SB-C18 column (4.6×150 mm, 3.5 μm particle size) with a gradient of solutions A (0.1% TFA in water) and B (0.1%TFA in ACN) at a flow rate of 1 ml/min: 25 min from 25% B to 54% B followed by a 32-min gradient from 54% B to 73% B. After UV and fluorescence detection, the flow was split 1:5 and then electrosprayed into the Exactive MS. For intact mass measurements the Exactive MS was operated with the following settings applied: spray voltage 4 kV, capillary temperature 275° C., sheath gas 20, aux gas 8, scan range 300-2,200 m/z, resolution ultra-high, AGC target 1e6, max inject time 100 ms, and microscans 10. Relative quantification of truncation products was performed based on the extracted ion chromatograms (EICs) of the native and the truncation products, respectively. Ion chromatograms were extracted in Xcalibur 2.1 using the theoretical masses of the +10 charged-molecules with a mass window of 0.5 Da.

Results are shown in FIG. 1 and additionally for two products in the table 1 below. The products contain between 0.7 and 1.3% group I truncations and between 0.5 and 0.6% group II truncations.

TABLE 1

| N-terminus of G-CSF and truncated | | Sample | Sample |
|---|---|---|---|
| variants | Class | 1 [%] | 2 [%] |
| Met Thr Pro Leu Gly Pro Ala Ser . . . | — | 98.7 | 98.2 |
| Thr Pro Leu Gly Pro Ala Ser . . . | Group I | 0.5 | 0.5 |
| Pro Leu Gly Pro Ala Ser . . . | Group I | 0.2 | 0.8 |
| Leu Gly Pro Ala Ser . . . | Group II | 0.0 | 0.0 |
| Gly Pro Ala Ser . . . | Group II | 0.1 | 0.1 |
| Pro Ala Ser . . . | Group II | 0.4 | 0.3 |
| Ala Ser . . . | Group II | 0.1 | 0.1 |
| Ser . . . | Group II | 0.0 | 0.0 |

### Example 2

### Analysis of the Abundance of N-Terminal G-CSF Truncation Products depending on the Concentration of the Nitrogen Source (NH₄)₂SO₄

Fermentations were conducted at 50-L scale according to the following principles: E. coli cells were cultivated in complex media containing inorganic salts like (NH₄)₂SO₄, glucose, and organic nitrogen sources at common fermentation temperature. Dissolved oxygen levels were kept above 30% by a cascade control of stirring, aeration, and back pressure and a neutral pH was maintained by titration with NH₄OH and H₂SO₄. After an initial batch growth phase, glucose feeding was started and the fed-batch phase initiated. Reaching a certain biomass content, cells were induced by the addition of IPTG. G-CSF expression was continued until harvest. To sustain both, growth and product expression, a continuous complex nutrient feed was applied. Different concentrations of (NH₄)₂SO₄, tested from 1 to 12.5 g/L in the cultivation medium, were evaluated in separate fermentations following aforementioned conditions. Upon fermentation, cells were harvested and RPC-MS analysis was conducted on the basis of isolated inclusion bodies. Data revealed a correlation between the ammonia level applied and the resulting truncation type II/III levels, see FIG. 2.

### Example 3

### Impact of NH₄OH Titration on Abundance of N-Terminal G-CSF Truncation Products

A fermentation was conducted according to the aforementioned procedure in Example 2 with a standard ammonia concentration (2 g/L (NH₄)₂SO₄). To allow for an elevated NH₄ concentration by pH titration with NH₄OH, the pH set-point was elevated by 0.2 units and glucose feeding was increased by 20%. As a consequence of the latter, the broth was acidified to a larger extent, which required elevated NH₄OH titration leading to higher ammonia contents in the broth throughout the fermentation. Truncation type II levels were reduced by 50% over a reference fermentation.

### Example 4

### Increasing Initial Nitrogen Concentration at the Start of Fermentation

To allow for higher ammonia levels at the onset of the fermentation without changing the medium composition, ammonia was added to the broth by shots of NH₄OH and titration by sulphuric acid before inoculation of the medium. Resulting truncation type II levels were lowered by up to 40%.

### Example 5

### Utilization of a Complex Nitrogen Solution

Fermentations were conducted according to the aforementioned procedure in Example 2 but applying a complex nutrient solution (yeast autolysate), which was supplemented by (NH₄)₂SO₄ in levels to reach 7.5 g/L (NH₄)₂SO₄ in the medium. Resulting truncation type II levels were in the range depicted by FIG. 2 for elevated ammonia levels (7.5 g/L (NH₄)₂SO₄).

### Example 6

### Bolus Addition of Nitrogen Source

In another fermentation a (NH₄)₂SO₄-bolus addition (7.5 g/L) was applied immediately after induction. Resulting truncation type II levels were in the range depicted by FIG. 2 for elevated ammonia levels (7.5 g/L (NH₄)₂SO₄).

### Example 7

### Yield Comparison for Human Recombinant G-CSF depending on the Concentration of Nitrogen Source Used

Based on principles of a purification process, in particular Cation Exchange (CEX) principles, truncated variants can be enriched in certain fractions. Such fractions are therefore prone to not meeting purity criteria and prone to being discarded. Fermentation batches with 7.5 or 12.5 g/L (NH₄)₂SO₄) were shown to avoid a loss of yield of up to 30% over reference batches. As illustrated in FIG. 4 showing truncation levels of the first CEX fractions not meeting the purity criterion, less truncated variants are present when elevated (NH₄)₂SO₄ levels are applied.

1. Method of producing a protein with reduced impurities resulting from truncation of said recombinant protein, the method comprising the steps of:
a) culturing host cells expressing the recombinant protein in a culture medium in presence of a nitrogen source in a concentration of said nitrogen source above standard,
b) isolating the protein, and
c) optionally purifying said isolated protein.

2. The method according to claim 1, wherein the nitrogen source level is increased to a concentration above standard by:
i) increasing the levels of the nitrogen source in the medium prior to cultivating the host cells in said medium,
ii) increasing NH₄OH during medium preparation and prior to sterilization;
iii) adding additional nitrogen source to a complex nutrient source and using said complex nutrient source when preparing the medium; and/or
iv) adding additional nitrogen source to the medium while already culturing the host cells.

3. The method according to claim 1, wherein the method further comprises the following step:
c) purifying the recombinant protein isolated from the host cells in step b).

4. The method according to claim 1, wherein the host cells are bacterial.

5. The method according to claim 1, wherein the nitrogen source is an inorganic nitrogen source.

6. The method according to claim 1, wherein the nitrogen source is an ammonium salt.

7. The method according to claim 1, wherein the nitrogen source is (NH₄)₂SO₄.

8. The method according to claim 1, wherein the protein is human granulocyte-colony stimulating factor (hG-CSF).

9. The method according to claim 8, wherein the reduced impurities are impurities resulting from truncated forms of hG-CSF according to SEQ ID NO:
3 lacking the first three, four, five, six or seven amino acids of SEQ ID NO: 3.

10. A composition comprising hG-CSF, in particular hG-CSF according to SEQ ID NO: 3, and further comprising less than 0.4% impurities resulting from group II truncations of hG-CSF according to SEQ ID NO: 3, said group II truncations of hG-CSF lacking the first three, four, five, six or seven N-terminal amino acids of SEQ ID NO: 3.

11. The composition according to claim 10, wherein the composition does comprise impurities resulting from group I truncations of hG-CSF according to SEQ ID NO: 3, said group I truncations of hG-CSF lacking the first 1 to 3 N-terminal amino acids of SEQ ID NO: 3.

12. The composition according to claim 11, wherein the ratio of group II truncations to group I truncations is less than 0.3.

13. The composition according to claim 10, wherein the composition is a cell lysate.

14. The composition according to claim 10 wherein the composition is a pharmaceutical composition comprising a pharmaceutically acceptable carrier.

15-16. (canceled)

17. A method of treatment of a subject suffering from neutropenia, the method comprising a step of administering a pharmaceutical composition according to claim 10 to said subject in an effective amount.

18. The method of treatment of claim 17, wherein the composition comprises hG-CSF.


SEQUENCE LISTING (19 sequences)

SEQ ID NO: 1 - protein, 175 aa, Artificial sequence
  Recombinant form of human G-CSF with N-terminal methionine
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 2 - protein, 174 aa, Homo sapiens
TPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 3 - protein, 175 aa, Artificial sequence
  Consensus sequence comprising sequence of natural human as well as recombinant human G-CSF
  MISC_FEATURE (1)..(1): Xaa may be Met or absent
XTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 4 - protein, 204 aa, Homo sapiens
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 5 - protein, 2 aa, Homo sapiens
TP

SEQ ID NO: 6 - protein, 3 aa, Homo sapiens
TPL

SEQ ID NO: 7 - protein, 4 aa, Homo sapiens
TPLG

SEQ ID NO: 8 - protein, 5 aa, Homo sapiens
TPLGP

SEQ ID NO: 9 - protein, 6 aa, Homo sapiens
TPLGPA

SEQ ID NO: 10 - protein, 3 aa, Artificial sequence
  3 N-terminal amino acids of recombinant G-CSF
MTP

SEQ ID NO: 11 - protein, 4 aa, Artificial Sequence
  4 N-terminal amino acids of recombinant G-CSF
MTPL

SEQ ID NO: 12 - protein, 5 aa, Artificial Sequence
  5 N-terminal amino acids of recombinant G-CSF
MTPLG

SEQ ID NO: 13 - protein, 6 aa, Artificial Sequence
  6 N-terminal amino acids of recombinant G-CSF
MTPLGP

SEQ ID NO: 14 - protein, 7 aa, Artificial Sequence
  7 N-terminal amino acids of recombinant G-CSF
MTPLGPA

SEQ ID NO: 15 - protein, 3 aa, Artificial sequence
  Consensus sequence comprising N-terminal sequence of natural human as well as recombinant human G-CSF
  MISC_FEATURE (1)..(1): Xaa may be Met or absent
XTP

SEQ ID NO: 16 - protein, 4 aa, Artificial Sequence
  Consensus sequence comprising N-terminal sequence of natural human as well as recombinant human G-CSF
  MISC_FEATURE (1)..(1): Xaa may be Met or absent
XTPL

SEQ ID NO: 17 - protein, 5 aa, Artificial Sequence
  Consensus sequence comprising N-terminal sequence of natural human as well as recombinant human G-CSF
  MISC_FEATURE (1)..(1): Xaa may be Met or absent
XTPLG

SEQ ID NO: 18 - protein, 6 aa, Artificial Sequence
  Consensus sequence comprising N-terminal sequence of natural human as well as recombinant human G-CSF
  MISC_FEATURE (1)..(1): Xaa may be Met or absent
XTPLGP

SEQ ID NO: 19 - protein, 7 aa, Artificial Sequence
  Consensus sequence comprising N-terminal sequence of natural human as well as recombinant human G-CSF
  MISC_FEATURE (1)..(1): Xaa may be Met or absent
XTPLGPA